(19) United States
(12) Patent Application Publication
(10) Pub. No.: US 2008/0038382 A1
(43) Pub. Date: Feb. 14, 2008

(54) Extracts of Scutellaria for the Treatment of Sars

(76) Inventors: Shouming Zhong, Oxford (GB); Hongwen Yu, Oxford (GB); Robert Miller, Oxon (GB)

Correspondence Address: DICKINSON WRIGHT PLLC, 38525 WOODWARD AVENUE, SUITE 2000, BLOOMFIELD HILLS, MI, 48304-2970, US

(21) Appl. No.: 10/590,287

(22) PCT Filed: Feb. 25, 2005
(86) PCT No.: PCT/GB05/00714

§ 371 Date: May 11, 2007

(30) Foreign Application Priority Data
Feb. 27, 2004 (GB) 040327.9

(51) Int. Cl. A61K 36/539; A61K 31/192; A61K 31/216; A61P 31/12; A61K 31/352; A61K 31/70

(52) U.S. Cl. 424/741; 424/725; 514/25; 514/456; 514/533; 514/570

(57) ABSTRACT

The present invention relates to pharmaceutical compositions having antiviral activity against Coronavirus, and more particularly against those viruses responsible for Severe Acute Respiratory Syndrome (SARS). In a preferred embodiment it comprises a total standardized extract of a Scutellariae spp.

### TECHNICAL FIELD OF THE INVENTION

The present invention relates to pharmaceutical compositions exhibiting antiviral activity. More particularly it relates to pharmaceutical compositions exhibiting antiviral activity against Coronavirus, and more particularly still against those viruses responsible for Severe Acute Respiratory Syndrome (SARS).

### BACKGROUND OF THE INVENTION

Recent observations with macaques (R. A. M. Fouchier, A. D. M. E. Osterhaus et al. Koch's postulates fulfilled for SARS virus Nature 423, 240 (2003).) and with a human cohort (T. Kuiken, A. D. M. E. Osterhaus, et al. Newly discovered Coronavirus as the primary cause of severe acute respiratory syndrome. Lancet 6318, 1 (2003)) provide conclusive evidence that a newly discovered Coronavirus (SARS-CoV) is the primary cause of Severe Acute Respiratory Syndrome (SARS), a form of viral pneumonia with a high mortality rate (˜10% globally (World Health Organisation—www.who.int/csr/sars)) that first arose in November 2002 in China.

Therefore, anti-viral agents with activity against SARS-CoV are likely to prove important in treating SARS. The Coronavirus genome consists of a single positive strand of RNA and the entire sequence of the SARS-CoV genome and related variants has been published (P. A. Rota et al. Characterization of a novel Coronavirus associated with Severe Acute Respiratory Syndrome, Science 300 1394 (2003) and M. A. Marra et al. The genome sequence of the SARS-associated Coronavirus, Science 300 1399 (2003)) and scrutinized for molecular targets for antiviral therapy. (http://www.sarsresearch.ca/—a bioinformatics site providing in depth data and tools to analyze the genomes, genes and proteins of SARS-CoV and related viruses.). Together with the viral polymerase enzyme, the main viral proteinase (3CLpro) appears to represent a key target (K. Anand et al. Coronavirus main proteinase (3CL^{pro}) structure: Basis for design of Anti-SARS drugs. Science 300 1763 (2003))

However, a good candidate drug (AG7088) failed to inhibit virus whilst apparently unrelated HIV therapies (Lopinavir, Nelfinavir) were partially active (SCRIP online—www.pjbpubs.co.uk/SCRIP/; Factiva Online—www.factiva.com/news).

In addition to known broad spectrum antivirals, e.g., ribavirin (RBV), less obvious inhibitors, e.g. glycyrrhizin, extracted from liquorice (Cinatl, J. et al. Glycyrrhizin, an active component of liquorice roots, and replication of SARS-associated Coronavirus. The Lancet, 361, 2045 (2003)) appear to be efficacious against the SARS-CoV.

However, well-founded concerns that an outbreak of SARS may re-occur has added impetus to a search for effective therapies.

Thus, there is a need for effective drugs and drug candidates for the treatment of SARS which are in dosage forms acceptable both to Eastern and Western patients.

### DEFINITIONS

In the specification the following definitions, taken from the U.S. Department of Health and Human Services, Food and Drug Administration, Center for Drug Evaluation and Research (CDER), August 2000 Guidance for Industry, Botanical Drug Products, are intended:

Active Constituent The chemical constituent in a botanical raw material, drug substance, or drug product that is responsible for the intended pharmacological activity or therapeutic effect.

Botanical Product; Botanical: A finished, labelled product that contains vegetable matter, which may include plant materials (see below), algae, macroscopic fungi, or combinations of these. Depending in part on its intended use, a botanical product may be a food, drug, medical device, or cosmetic.

Botanical Drug Product; Botanical Drug: A botanical product that is intended for use as a drug; a drug product that is prepared from a botanical drug substance. Botanical drug products are available in a variety of dosage forms, such as solutions (e.g., teas), powders, tablets, capsules, elixirs, and topicals.

Botanical Drug Substance: A drug substance derived from one or more plants, algae, or macroscopic fungi. It is prepared from botanical raw materials by one or more of the following processes: pulverization, decoction, expression, aqueous extraction, ethanolic extraction, or other similar process. It may be available in a variety of physical forms, such as powder, paste, concentrated liquid, juice, gum, syrup, or oil. A botanical drug substance can be made from one or more botanical raw materials (see Single-Herb and Multi-Herb botanical drug substance or product). A botanical drug substance does not include a highly purified or chemically modified substance derived from natural sources.

Botanical Ingredient: A component of a botanical drug substance or product that originates from a botanical raw material.

Botanical Raw Material: Fresh or processed (e.g., cleaned, frozen, dried, or sliced) part of a single species of plant or a fresh or processed alga or macroscopic fungus.

Chromatographic Fingerprint: A chromatographic profile of a botanical raw material or drug substance that is matched qualitatively and quantitatively against that of a reference sample or standard to ensure the identity and quality of a batch and consistency from batch to batch.

Dietary Supplement: [A] product (other than tobacco) intended to supplement the diet that bears or contains one or more of the following dietary ingredients: (A) a vitamin; (B) a mineral; (C) an herb or other botanical; (D) an amino acid; (E) a dietary substance for use by man to supplement the diet by increasing the total dietary intake; or (F) a concentrate, metabolite, constituent, extract, or combination of any ingredient described in clause (A), (B), (C), (D), or (E); (2) means a product that (A) is intended for ingestion in a form described in section 411(c)(1)(B)(i) [of the FD&C Act]; or complies with section 411(c)(1)(B)(ii); is not represented for use as a conventional food or as a sole item of a meal or the diet; and is labeled as a dietary supplement; and (3) does (A) include an article that is approved as a new drug under section 505 or licensed as a biologic under section 351 of the Public Health Service Act (42 U.S.C. 262) and was, prior to such approval, certification, or license, marketed as a dietary supplement or as a food unless [FDA] has issued a regulation, after notice and comment, finding that the article, when used as or in a dietary supplement under the conditions of use and dosages set forth in the labeling for such dietary supplement, is unlawful under section 402(f); and (B) not include (i) an article that is approved as a new drug under section 505, certified as an antibiotic under section 507, or licensed as a biologic under section 351 of the Public Health Service Act (42 U.S.C. 262), or (ii) an article authorized for investigation as a new drug, antibiotic, or biological for which substantial clinical investigations have been instituted and for which the existence of such investigations has been made public, which was not before such approval, certification, licensing, or authorization marketed as a dietary supplement or as a food unless [FDA], in [its] discretion, has issued a regulation, after notice and comment, finding that the article would be lawful under this Act_(21 U.S.C. 321(ff)).

Dosage Form: A pharmaceutical product type, for example, tablet, capsule, solution, or cream, that contains a drug ingredient (substance) generally, but not necessarily, in association with excipients.

Drug: Means (A) articles recognized in the official United States Pharmacopeia, official Homeopathic Pharmacopeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals; and (D) articles intended for use as a component of any articles specified in clause (A), (B), or (C). A food or dietary supplement for which a claim, subject to sections 403(r)(1)(B) and 403(r)(3) [of the FD&C Act] or sections 403(r)(1)(B) and (r)(5)(D), is made in accordance with the requirements of section 403(r) is not a drug solely because the label or the labeling contains such a claim. A food, dietary ingredient, or dietary supplement for which a truthful and not misleading statement is made in accordance with section 403(r)(6) is not a drug under clause (C) solely because the label or the labeling contains such a statement_(21 U.S.C. 321(g)(1)).

Drug Substance: An active ingredient that is intended to furnish pharmacological activity or other direct effect in the diagnosis, cure, mitigation, treatment, or prevention of disease or to affect the structure or any function of the human body (21 CFR 314.3(b)).

Drug Product: The dosage form in the final immediate packaging intended for marketing.

Food: The term food means (1) articles used for food or drink, (2) chewing gum, and (3) articles used for components of such articles (21 U.S.C. 321(f)).

Formulation: A formula that lists the components (or ingredients) and composition of the dosage form. The components and composition of a multi-herb botanical drug substance should be part of the total formulation.

Marker: A chemical constituent of a botanical raw material, drug substance, or drug product that is used for identification and/or quality control purposes, especially when the active constituents are not known or identified.

Multi-Herb (Botanical Drug) Substance or Product: A botanical drug substance or drug product that is derived from more than one botanical raw material, each of which is considered a botanical ingredient. A multi-herb botanical drug substance may be prepared by processing together two or more botanical raw materials, or by combining two or more single-herb botanical drug substances that have been individually processed from their corresponding raw materials. In the latter case, the individual single-herb botanical drug substances may be introduced simultaneously or at different stages during the manufacturing process of the dosage form.

Plant Material: A plant or plant part (e.g., bark, wood, leaves, stems, roots, flowers, fruits, seeds, berries, or parts thereof) as well as exudates.

Single-Herb (Botanical Drug) Substance or Product: A botanical drug substance or drug product that is derived from one botanical raw material. Therefore, a single-herb substance or product generally contains only one botanical ingredient.

In addition the terms:

Consisting essentially is intended to refer back only to the presence of botanical raw materials and their derivatives and excludes the presence of e.g. excipients used in the formulation; and

Treatment is intended to refer to both symptomatic relief and/or activity against the causative factor

### DISCLOSURE OF THE INVENTION

Surprisingly, the applicant has found that a composition (PYN5C) shows dose dependant activity against SARS-CoV in cell culture tests.

According to a first aspect of the present invention there is provided the use of a botanical raw material (BRM), a botanical drug substance (BDS), or one or more botanical ingredients, obtainable from a species of the genus Scutellaria in the manufacture of an anti viral medicament.

Preferably, the medicament is for the treatment of patients infected by a positive strand RNA virus, more particularly SARS-CoV.

In one embodiment the medicament consists essentially of a single botanical drug substance or botanical ingredient.

Preferably the medicament is formulated with excipients. In one embodiment the medicament is in a suspension dosage form.

According to a second aspect of the present invention there is provided a suspension dosage form medicament comprising a Scutellariae spp.

It will however be apparent that any suitable dosage form will be acceptable e.g. a solid dosage form, such as a tablet or a liquid dosage form, such as a syrup.

Similarly the medicament may be formulated for delivery by any route e.g. orally, intravenously or by any other recognised form.

In a favoured embodiment, since the medicament is for treating upper respiratory tract infections, it is formulated for delivery as a spray, and more particularly as a nebuliser.

In another embodiment the medicament comprises, in addition to the Scutellaria spp, one or more additional botanical drug substances or botanical ingredients obtainable from one or more of a:

b) Lonicera spp;

c) Forsythia spp; and/or

d) Rabdosia spp;

PYN5C is an ethanolic single herb extract of Radix Scutellariae.

An aqueous extract of a Scutellaria spp has been shown to exhibit antiviral activity against Picornaviridae (polio—a negative strand RNA virus) and Paramyxoviridae (measles—another negative strand RNA virus) see WO 5,411,733.

Plant flavenoids (including baicalein, baicalin and wogonin) isolated from Scutellaria spp, have also been shown to exhibit antiviral activity (primarily, though not exclusively, against HIV and have also been shown to demonstrate activity against RSV (Journal of Ethnopharmacology (2002) 79 (2), p 205-211) and influenza.

In spite of the above, the finding that PYN5C showed activity against SARS-CoV was unexpected, as generally speaking the plant is used in Chinese medicine for it's antibacterial activity and is furthermore typically used in combination with several other plant species. Indeed the applicant was surprised that this single plant extract showed activity although they hoped a combination comprising, for example, extracts of three herbs Radix Scutellariae, Fructus Forsythiae and Flos Lonicerae, a composition not dis-similar to a licensed Chinese medicine Shang Huang Lian (SHL) might prove to be effective against SARS-CoV

Thus, according to third aspect of the present invention there is provided the use of one or more botanical raw materials (BRM), one or more botanical drug substances (BDS), or one or more botanical ingredients obtainable from a species of the genus:

a) Scutellaria

b) Lonicera;

c) Forsythia; or

d) Rabdosia

in the manufacture of a botanical drug (BD), or dietary supplement for the treatment of a patient infected with SARS-CoV.

Any suitable species from the above plant genera may be used. These include:

a) as Scutellaria spp: Scutellaria baicalensis, S. amoena, S. barbata, S. discolor, S. hypericifolia, S. inbica, S. likiangensis, S. orthocalyx, S. rehderiana, S. scssiliflora and S. viscidula;

b) as Lonicera spp: Lonicera japonica, L. hispidapall, L. harmsii, and L. fulvotomentosa;

c) as Forsythia spp: Forsythia suspensa, F. viridissima, F. ovata, F. mandschurica, F. koeana, F. spectabilis, F europaea, and F. X intermedia; and

d) as Rabdosia spp Rabdosia rubescens, R. adenantha, R. amethystoides, R. coetsa, R. nervosa, R. sculponeata and R. ternioflia.

A botanical drug may be obtained with a combination of these species, particularly, though not exclusively, a combination of either:

a) Scutellaria baicalensis;

b) Lonicera japonica;

c) Forsythia suspensa and

d) Rabdosia rubescens.

Preferred combinations comprise, consist essentially of or consist of one or a combination of a species from each genera, particularly one or more of the preferred species identified above.

Preferred combinations include, but are not limited to, the combinations of a species of the genera (and the preferred species identified above) as set out below:

1. a) Scutellaria spp and c) Forsythia spp;

2. a) Scutellaria spp and b) Lonicera spp;

3. a) Scutellaria spp and d) Rabdosia spp;

4. a) Scutellaria spp, b) Lonicera spp and c) Forsythia spp.

5. a) Scutellaria spp, b) Lonicera spp c) Forsythia spp and d) Rabdosia spp.

Preferably each species is present as a botanical drug substance or a botanical ingredient.

Any suitable part of the plants can be used. For example leaves, twigs, branches, bark, roots, flowers and fruits can be used.

The preferred part of a favoured species of the genus:

a) Scutellaria is the root;

b) Lonicera is the flower;

c) Forsythia is the fruit; and

d) Rabdosia is the aerial parts, i.e. any part other than the root;

The relative amount of each species (calculated as dry weight of botanical raw material) will vary depending on the given combination.

For single herb medicaments and combination herb medicaments the amount of each botanical (calculated as dry weight of botanical raw material) will typically be in the range shown in table 1 below:

TABLE 1

| | Daily dose of | Two herb | Three herb | Four herb |
|---|---|---|---|---|
| | single herb product | product | product | product |
| a) Lonicera spp | 6-15 g/day | 25-75% of daily | 12.5 to 37.5%, of daily | 5-15% of daily |
| | dry herb | dose typically 50% | dose typically 27.5%. | dose typically 10% |
| b) Forsythia spp | 6-15 g/day | 25-75% of daily | 30 to 70% of daily | 12.5-37.5% of daily |
| | dry herb | dose typically 50% | dose typically 55% | dose typically 25% |
| c) Scutellaria spp | 3-9 g/day | 25-75% of daily | 12.5 to 37.5%, of daily | 22.5-7.5% of daily |
| | dry herb | dose typically 50% | dose typically 27.5%. | dose typically 55% |
| d) Rabdosia spp | 30-60 g/day | 25-75% of daily | | 5-15% of daily |
| | dry herb | dose typically 50% | | dose typically 10% |

For dual combinations each plant may be present to provide from 5-95% of the botanical content, more preferably from 25-75% and most preferably from 40-60%.

In the case of a three botanicals combination the amounts may vary depending on the combination.

Where the combination consists essentially of a Scutellaria spp; Lonicera spp; and Forsythia spp; each species may be present in the following amounts:

Forsythia spp; is present in an amount by weight relative to the total weight of all the botanical raw materials of from 30 to 70%, more preferably 40 to 60% and most preferably 50% or more, most preferably greater than 55%

Lonicera spp; is present in an amount by weight relative to the total weight of all the botanical raw materials of from 12.5 to 37.5%, more preferably 18.75 to 31.25% and most preferably about 27.5%.

Scutellaria spp; is present in an amount by weight relative to the total weight of all the botanical raw materials or ingredients of from 12.5 to 37.5%, more preferably 18.75 to 31.25% and most preferably about 27.5%.

According to a forth aspect of the present invention there is provided a suspension powder mixture comprising as botanicals:

a Forsythia spp in an amount by weight relative to the total weight of all the botanical raw materials of from 30 to 70%,

a Lonicera spp in an amount by weight relative to the total weight of all the botanical raw materials of from 12.5 to 37.5%, and

a Scutellaria spp in an amount by weight relative to the total weight of all the botanical raw materials of from 12.5 to 37.5% and as excipients:

one or more gellants or thickeners comprising at least one xanthum gum having a particle size distribution such that 100% by weight of the particles pass a 60 mesh sieve, 95% by weight of the particles pass a 80 mesh sieve and 70% by weight of the particles pass a 200 mesh sieve, one or more fillers; and one or more wetting agents or surfactants.

The applicant has reason to believe that species of the genera Forsythia may, in addition to the Scutellaria spp demonstrate activity against SARS-CoV.

Thus, in another embodiment the Forsythia comprises greater than 50% of the total plant extracts (by weight of the botanical raw material equivalents).

Where the Scutellaria spp or Forsythia spp is demonstrated to be the primary active it is preferred that it comprises greater than 50% of the plant components, more preferably greater than 60% more preferably still greater than 70%, through 80 and 90% to as much as 100%.

The invention also extends to a method of treating patients infected with SARS-CoV by administering a medicament according to the invention to the patient.

### BRIEF DESCRIPTION OF DRAWING

The single FIGURE is a digital image of representative assay plates demonstrating the inhibitory effect of PYN5C against SARS-CoV.

### BEST MODE FOR CARRYING OUT THE INVENTION

The claimed invention is based on the finding that PYN 5C, a lyophilised 70% ethanolic extract of a Scutellaria spp inhibited SARS-CoV in cell culture.

By reference to what is known about:
i) the composition of SHL and similar herbal combinations;
ii) the presumed actives of Scutellaria spp, Lonicera spp, Forsythia spp and Rabdosia spp; and
iii) alternative Chinese herbs providing similar medicinal effects in Traditional Chinese Medicine
   the applicant, by way of extrapolation, proposes that in addition to their, Scutellaria extract different extracts to the one they have initially tested, as well as alternative herbal materials or their identifiable botanical ingredients or active constituents, may be responsible for the SARS-CoV inhibitory activity and may additionally prove useful in treating other viral infections, particularly RNA viruses and more particularly positive RNA stranded viruses including, for example, RSV, influenza and Avian Flu.

Thus, for example, in U.S. Pat. No. 6,083,921, the contents of which document is incorporated by reference, it is suggested that:

a) Baicalin isolated from Radix Scutellariae;

b) Chlorogenic acid isolated from Flos Lonicerae and

c) Forsythiaside isolated from Fructus Forsythiae

are the active components of SHL.

More particularly, U.S. Pat. No. 6,083,921, teaches a first composition comprising:

a) 0.25 mg Radix Scutellariae;

b) 0.25 mg Fructus Forsythiae; and

c) 0.5 mg Flos Lonicerae per ml of composition

and a second composition comprising:

a) 2 mg baicalin;

b) 1 mg chlorogenic acid; and

c) 1 mg forsythiaside per ml of composition.

Thus, the applicant hypothesises that such formulations may, like applicants composition, show activity against SARS-CoV.

Thus, according to a fifth aspect of the present invention there is provided any one of, or any combination of Baicalin, Chlorogenic acid and Forsythiaside for use in the manufacture of a drug or dietary supplement for the treatment of SARS-CoV

Furthermore, in U.S. Pat. No. 6,083,921 it is suggested that a number of closely related compounds, namely the compounds of Formulae I and Formulae II as identified in column 2 therein may possess anti viral activity.

Applicants predict that these compounds may also show activity against SARS-CoV.

Other work on improved SHL like compositions is disclosed in WO 02/060379, the contents of which document is also incorporated by reference. In WO 02/060379 an improved SHL tablet is disclosed. It is made from different and “improved” extracts of:

a) Flos Lonicerae;

b) Fructus Forsythiae; and

c) Radix Scutellariae.

More particularly, the extraction methods used give rise to defined drug substances and drug products which it is claimed are more efficacious in the inhibition of influenza virus, parainfluenza virus, herpes virus I and herpes virus II. The extraction method used to obtain these more active fractions is supercritical carbon dioxide extraction.

Thus, accordingly the applicants predict that supercritical carbon dioxide extracts may show greater activity than their ethanolic extracts.

The specific formula disclosed in WO 02/060379 comprises a ratioed mix of the raw herbal materials in amounts of:
a) Flos Lonicerae 1 part by weight (equivalent to 1875 g of raw material);
b) Fructus Forsythiae 2 parts by weight (equivalent to 3750 g of raw material); and
c) Radix Scutellariae 1 part by weight (equivalent to 1875 g of raw material).

More particularly it comprises
i) 90-180 parts a soft extract of Flos Lonicerae and Fructus Forsythiae;
ii) 10-60 parts of a supercritical extract of Flos Lonicerae and Fructus Forsythiae; and
iii) 30-50 parts of an extract of Radix Scutellariae.

In the specification 3d spectro chromatograms are used to characterise the extracts:

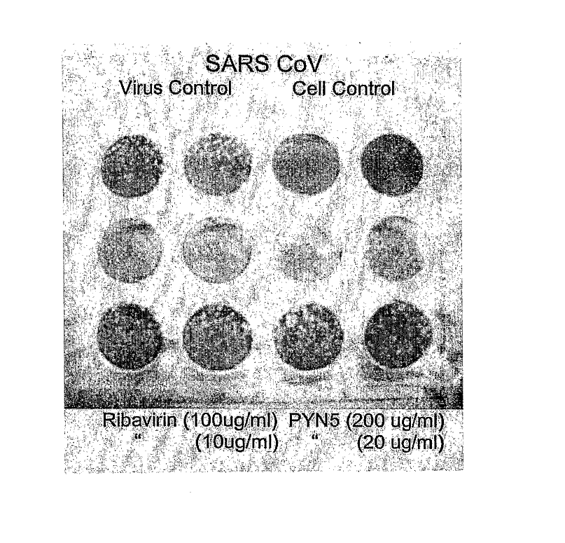

Flos Lonicerae raw material is shown to have 8-11 peaks, the 4^{th} peak of which is Chlorogenic acid which is used as a reference peak (FIG. 2 of the specification);

Fructus Forsythiae raw material is shown to have 11-14 peaks, the 8^{th} peak of which is Phillyrin which is used as a reference peak (FIG. 3 of the specification); and

Radix Scutellariae raw material is shown to have 22-25 peaks, the 12^{th} peak of which is Baicalin and the 21^{st} peak is Baicalein both of which are used as reference peaks (FIG. 4 of the specification).

Furthermore the extracts showing improved efficacy differ in content from the raw materials due to the extraction techniques employed.

Thus, Flos Lonicerae and Fructus Forsythiae were subjected to extraction together and the extract was shown to have 18 to 21 peaks, the 8^{th}, 10^{th} and 16^{th} of which were reference peaks for Chlorogenic acid, Caffeic acid and Phillyrin respectively (FIG. 5 of the specification);

Radix Scutellariae had 4-5 peaks the 1^{st} of which was Baicalin and the 5^{th} of which was Baicalein (FIG. 6 of the specification); and

The drug product which consisted of extracts of the 3 herbs had 27-30 peaks of which the 8^{th}, 12^{th}, 20^{th}, 22^{nd} and 28^{th} were Chlorogenic acid, Caffeic acid, Phillyrin, Baicalin and Baicalein respectively (FIG. 7 of the specification).

Applicant surmises that, based on the activity they have demonstrated, these compositions and similar ones might also be expected to show activity.

In a preferred embodiment of the applicant's invention the dosage form is a suspension powder, typically packaged in a sachet, preferably with a container for preparing the unit dose for oral administration. The container preferably holds a volume of less than 100 ml, and is preferably marked such that the user knows how much liquid to add in order to suspend the product. Most preferably the container is provided with a sealable lid so that it can be vigorously shaken such that the medicine is suspended.

The preferred excipients of the suspension powder include:

a) one or more gellants or thickeners, preferably comprising at least one xanthum gum having a particle size distribution that 100% by weight of the particles pass a 60 mesh sieve, 95% by weight of the particles pass a 80 mesh sieve and 70% by weight of the particles pass a 200 mesh sieve,

b) one or more fillers, particularly taste masking agents; and

c) one or more wetting agents or surfactants or other agents which aid suspension.

Suitable materials are described in EP 1231746 which is incorporated by reference.

Most preferably the dosage form is suspendable in a cold solvent, such as water, and in a volume of less than 50 ml, more preferably less than 25 ml.

A preferred xanthan gum has a molecular weight of 3.5 to 4.0×10⁶ such as that sold as Ferwogel.

A preferred wetting agent is a polyethylene glycol or macrogol.

The botanical drug substance or dietary supplement may additionally comprise one or more of a disintegrating agent, lubricant, sweetening agent, flavouring agent and a viscosifying agent.

An example of excipients that may be used in formulating the botanical drug substance(s) are shown in table 2 below:

TABLE 2

| General | | | Specific example |
|---|---|---|---|
| components | Specific examples | General ranges | (for unit dose) |
| Wetting agent | Macrogol 6000 | 0.1 to 50% | 0.600 g |
| or surfactant | powder | | |
| Gellant/ | Xanthan gum- | 0.01 to 80% | 0.070 g |
| thickener | Polysorbate | by weight | |
| | (Ferwogel 30.385) | | |
| Filler, | Mannitol | 10 to 75% | 0.160 g |
| preferably a | (Mannitol EZ) | by weight | |
| sugar or sugar | | | |
| alcohol | | | |
| Optionally | Colloidal Silicon | | 0.050 g |
| drying agent/ | dioxide | | |
| flowability | (Aerosil 200) | | |
| agent | | | |
| Optionally a | Pepermint powder | | 0.060 |
| flavouring | aroma | | |
| agent (taste | | | |
| masking agent) | | | |
| Optionally a | Aspartame | | 0.050 g |
| sweetener | | | |
| Optionally a | Caramel powder | | 0.100 g |
| colorant | (Colorant E150-a) | | |

The invention is further described, by way of example only, with reference to the following examples.

### EXAMPLE 1

### Extraction Protocol

The Scutellaria spp was subjected to an extraction process as set out below:
1. Grind material to a fine powder;
2. Weigh 100 g of coarse powder and extract it under reflux for 2 hours using 1 litre of 70% ethanol;
3. When cool, filter through a filter paper and collect the ethanol extract;
4. To the Scutellaria spp residue, add 1 litre of 70% ethanol and reflux for 2 hours and repeat as step 3;
5. Combine the ethanol extracts of steps 3 and 4;
6. Recover the solvent on a “RotaVapour” to a small volume suitable for lyophilising;
7. Lyophilise the extract;
8. Weigh the lyophilised extract and store in sealed glass containers.
   Test for Activity Against Coronavirus

PYN 5C was tested against two viruses (SARS CoV and RSV S2, supplied by NCPV) in cell culture (vero C1008 cells, originally supplied by ECACC). Each test material was made up in DMSO and added to the culture overlay at two different final concentrations. After 3 days incubation at 37° C., the cell monolayers were fixed stained and any plaques counted.

The results using RSV2s2 were inconclusive. No plaques were produced although there was a marginal difference in the monolayers comparing the cell and virus controls. The virus clearly grew producing syncytia but the assay possibly required a longer incubation period to allow cell death and visible plaques. Neither test material appeared to protect the cells (except possibly Ribavirin at 10 μg/ml) although the 100 μg/ml concentration did not protect.

Surprisingly the results using SARS-CoV yielded clearly defined plaques (the number of plaques are tabulated in table 3 below) and photographs of the representative plates are shown in the single FIGURE.

TABLE 3

| Virus control | Cell control |
|---|---|
| 113, 121, 111, 126 | 0, 0, 0, 0 |
| mean 117.5 | mean 0 |
| (100%) | (0%) |
| Ribavirin (100 μg/ml) | PYN5C (200 μg/ml) |
| 109, 79, 77, 84 | 58, 53, 50, 44 |
| mean 87.25 | mean 51.25 |
| (74.1%) | (43.15%) |
| Ribavirin (10 μg/ml) | PYN5C (20 μg/ml) |
| 125, 120, 116, 98 | 88, 102, 93, 89 |
| mean 114.75 | mean 93.00 |
| (97.5%) | (79.0%) |

For both the higher concentrations of Ribavirin and PYN5C the plaque size was smaller than for the controls.

### CONCLUSION

The PYN5C composition inhibits SARS-CoV infectivity approximately 50% at the highest concentration used (200 μg/ml). The effect would appear to be dose dependant given that there is less inhibition at the lower dose trialled. Significantly the inhibition at the higher level was greater than Ribavirin (100 μg/ml).

### EXAMPLE 2

The herb extract was formulated into a suspension dosage form by mixing with the following excipients:

Excipients:

| | |
|---|---|
| Macrogol 6000 powder | 0.600 g |
| Ferwogel 30.385 | 0.070 g |
| Mannitol EZ | 0.160 g |
| Aerosil 200 | 0.050 g |
| Aspartame | 0.050 g |
| Caramel powder | 0.100 g |
| Pepermint powder aroma | 0.060 g |

1. A method for the treatment of a person afflicted with SARS-CoV infection, comprising a single botanical drug substance or one or more botanical ingredients, obtained from a species of the genus Scutellaria selected from the group consisting of Scutellaria baicalensis, S. amoena, S. barbata, S. discolor, S. hypericifolia, S. inbica, S. likiangensis, S. orthocalyx, S. rehderiana, S. scssiliflora and S. viscidula in the manufacture of a medicament for the treatment of a patient with a SARS-CoV infection.

2. The method recited in claim 1, wherein the medicament is a total extract of a Scutellaria spp.

3. The method recited in claim 1, in which the medicament further comprises one or more excipients.

4. The method recited in claim 1, wherein the substance administered is a botanical drug and is a standardised extract.

5. The method recited in claim 4, wherein the botanical drug substance from the Scutellaria spp is standardised against a marker of baicalin and/or baicalein.

6. The method recited in claim 4, wherein the standardised extract is a dried ethanolic extract.

7. The method recited in claim 4, wherein the standardised extract is a lyophilised extract.

8. The method recited in claim 1, wherein the medicament is a botanical drug.

9. The method recited in claim 8, wherein the botanical drug is packaged in a sachet.

10. The method recited in claim 9, wherein the botanical drug is packaged with a dispensing container.

11. The method recited in claim 10, wherein the dispensing container has a sealable lid.

12. The method recited in claim 3, wherein the excipients comprise one or more gellants or thickeners comprising at least one xanthum gum having a particle size distribution such that 100% by weight of the particles pass a 60 mesh sieve, 95% by weight of the particles pass a 80 mesh sieve and 70% by weight of the particles pass a 200 mesh sieve; one or more fillers; and one or more wetting agents or surfactants.

13. The method recited in claim 1, wherein the one or more botanical ingredients obtained from a species of the genus Scutellaria are plant flavenoids or synthetic equivalents thereof.

14. The method recited in claim 13, wherein the plant flavenoids are selected from the group consisting of baicalein, baicalin and wogonin.

15. The method of making a medicament, effective as an anti-viral treatment, comprising admixing a botanical raw material, a botanical drug substance or one or more botanical ingredients, obtainable from a species of the genus Scutellaria.

16. The method recited in claim 15, wherein the antiviral medicament is effective for the treatment of a patient with a positively stranded RNA viral infection.

17. The method recited in claim 15, wherein the medicament is effective for the treatment of a patient with a SARS-CoV infection.

18. A suspension dosage medicament comprising a Scutellaria spp.

19. The use of one or more botanical raw materials, one or more botanical drug substances, or one or more botanical ingredients obtainable from a species of the genus:
(a) Scutellaria;
(b) Lonicera;
(c) Forsythia; or
(d) Rabdosia
in the manufacture of a botanical drug, or dietary supplement effective for the treatment of SARS-CoV.

20. A suspension powder mixture of botanical raw materials effective as an anti-viral medicament, comprising:
Forsythia in an amount by weight relative to the total weight of all the botanical raw materials of from 30 to 70%,
Lonicera in an amount by weight relative to the total weight of all the botanical raw materials of from 12.5 to 37.5%, and
Scutellaria in an amount by weight relative to the total weight of all the botanical raw materials of from 12.5 to 37.5% and:
one or more gellants or thickeners comprising at least one xanthum gum having a particle size distribution such that 100% by weight of the particles pass a 60 mesh sieve, 95% by weight of the particles pass a 80 mesh sieve and 70% by weight of the particles pass a 200 mesh sieve; one or more fillers; and one or more wetting agents or surfactants.

21. The suspension powder mixture as claimed in claim 20 comprising standardised extracts of each of the Forsythia, Lonicera and Scutellaria species.

22. The suspension powder mixture as recited in claim 21 wherein:
the Forsythia spp is standardised against a marker of Phillyrin;
the Scutellaria spp is standardised against a marker of either or both of Baicalin and Baicalein, and the
Lonicera spp is standardised against a marker of Chlorogenic acid and/or caffeic acid.

23. A method of making a medicament for treating SARS-CoV, comprising the use of one or a plurality of baicalin, baicalein, chlorogenic acid, forsythiaside, caffeic acid and phillyrin in a medicament effective for use in the treatment of SARS-CoV.

24. A medicament effective as an anti-viral agent consisting essentially of: botanical drug substances or botanical ingredients obtainable from a species of:
(a) Scutellaria;
(b) Lonicera;
(c) Forsythia; and
(d) Rabdosia

25. A method of treating SARS-CoV comprising administering to a patient a medicament as claimed in claim 24.
